(19) United States
(12) Patent Application Publication
(10) Pub. No.: US 2023/0157518 A1
(43) Pub. Date: May 25, 2023

(54) MEDICAL DEVICE AND ENDOSCOPIC METHOD

(71) Applicant: Lutz FREITAG, Hemer (DE)

(72) Inventor: LUTZ FREITAG, Hemer (DE)

(21) Appl. No.: 17/919,914

(22) PCT Filed: Apr. 21, 2021
(86) PCT No.: PCT/EP2021/060368

§ 371 Date: Oct. 19, 2022

(30) Foreign Application Priority Data
Apr. 21, 2020 (DE) 10 2020 110 845.1

(51) Int. Cl. A61B 1/00

(52) U.S. Cl. CPC A61B 1/00066; A61B 1/00128; A61B 1/00105

(57) ABSTRACT

A medical device includes an endoscope head which is designed to be detachably connected to endoscope tubes. The endoscope head has a longitudinal channel for receiving an endoscope tube. The longitudinal channel has a variable opening width for receiving endoscope tubes with different diameters. The endoscope head includes a clamping device in order to hold an endoscope tube inserted into the longitudinal channel in a clamped manner

The invention relates to a medical device having the features of patent claim 1 and to an endoscopic method according to the features of patent claim 12.

Endoscopes are used for minimally invasive surgical procedures on humans and animals as well as cavities which are difficult to access in the art for visual inspection. Depending on the intended use, endoscope tubes with different diameters find application. In bronchoscopy, rigid bronchoscope tubes of stainless steel with wall thicknesses of 0.6 mm are oftentimes used. Diameters in the range of 10 mm with lengths of approximately 400 mm are customary. For placing the frequently used silicone stents, tube diameters of 10-16 mm are required according to the width of a normal larynx. Handling with thicker equipments for stent insertion requires practice and experience.

A further problem is that in endoscopy, it is normally not possible to change from a smaller diameter to a larger diameter (or vice versa) without greater effort. For this purpose, it normally requires to completely exchange the endoscope tube including the endoscope head attached thereto and to reattach all the connections arranged therein.

The invention is based on the object to provide a medical device with an endoscope head which enables the use endoscope tubes of different diameters in a simple manner. It is intended to demonstrate a suitable method that entails the stated advantages.

The subject part of the object is achieved by a medical device having the features of patent claim 1.

A method having the features of claim 12 attains the part of the object relating to the method.

The respective subclaims relate to advantageous refinements of the invention.

The medical device includes an endoscope head which is designed to be detachably connected to endoscope tubes. For this purpose, the endoscope head has a longitudinal channel for receiving an endoscope tube.

The invention is not concerned with the subject matter of providing a non-detachable connection between the endoscope head and the endoscope tube, but relates predominantly to the design of the endoscope head, which, for example, is provided after connection to the endoscope tube and after completion of the examination or treatment, to be separated again from the endoscope tube.

The special feature of the endoscope head is that the longitudinal channel has at least in one region a variable opening width for receiving endoscope tubes of different diameters. Unlike endoscope heads, which can be connected, for example, to endoscope tubes of different lengths but have a uniform diameter, the endoscope head has a variable opening width. The opening width can be conformed to the diameter of the endoscope tubes to be received.

Furthermore, the endoscope head includes a clamping device in order to hold an endoscope tube inserted into the longitudinal channel in a damping manner. The clamping device has two objectives: firstly, it should hold the endoscope tube in a clamping manner, and secondly it is intended to match the opening width of the longitudinal channel in the endoscope head to the respective diameter. The clamping device is further preferably provided to hold the endoscope tube tightly and in particular in a gas-tight manner in the longitudinal channel. This can be realized by incorporating sealing means which seal a gap between the endoscope tube and the endoscope head.

In the non-loaded state, i.e. in the non-clamped state, the clamping device can assume an open position, i.e., clear the maximum opening width. Clamping of the clamping device for generating the clamping action is established via a force introduction from outside upon the damping device, so that the opening width decreases.

In an advantageous configuration of the invention, the clamping device has several displaceable clamping bodies. It involves preferably at least three clamping bodies, which are arranged in particular evenly dispersed over the circumference of the longitudinal channel The several displaceable clamping bodies enable the endoscope tube to be centered in the longitudinal channel

The clamping bodies can be forced or urged from radially outwards against the received endoscope tube. The clamping action is effected through radial pressure which is exerted from outside onto the wall of the endoscope tube via the clamping bodies. The clamping bodies, arranged in particular evenly dispersed over the circumference, are preferably all displaced uniformly, and have in particular the same geometry.

The clamping bodies can be separate components which are not connected to one another and which are guided in a guide cage and are held captivated in this cage.

It is considered advantageous to movably connect the clamping bodies to a common base body. As a result, the clamping bodies are coupled captivated to the base body. A guide cage is not required.

The base body itself can be a component of the endoscope head or be connected thereto. The clamping bodies, the base body and also the entire endoscope head are preferably produced of same material in one piece, for example from suitable plastic. The material properties can vary depending on the function of the individual regions. For example, the individual damping bodies can be made of a more flexible material in the transition zone to the base body to which they are secured and made of less flexible material in those regions in which the force must be transmitted' between the endoscope tube and the endoscope head. The desired mobility of the clamping bodies relative to the common base body can be achieved, in particular, by a geometric design that best conforms to demands, for example by designing movable regions that serve as joint or hinge to be slightly thinner.

The clamping bodies are in particular spring tongues, in particular those which are secured on one side and therefore have a free, movable end which can be urged against the endoscope tube. The displacement of the damping bodies for damping can be implemented through engagement in a run-on surface of a damping sleeve which can be displaced axially with respect to the base body. Such a clamping sleeve, which can also be referred to as a sliding sleeve, could have a constant inner diameter, while the individual clamping bodies have an outer diameter, which increases in longitudinal direction and hereby becomes greater than the inner diameter of the clamping sleeve. When viewed in longitudinal direction, the cross section of the clamping bodies can be wedge-shaped. When the clamping sleeve with constant diameter is pushed over the wedge-shaped clamping bodies, the clamping sleeve comes into contact with the clamping bodies. The clamping bodies are urged inwards. The clamping device is tensioned. An endoscope tube is held in a clamping manner.

Conversely, it is also possible to use a clamping sleeve with a run-on surface which tapers in the shape of a funnel, in particular conically, while the outer diameter of the clamping bodies remains the same. In this case, the clamping sleeve with the tapering, in particular conical, run-on surface is moved over the clamping bodies according to the wedge principle in order to force it inwards or to release it again when the clamping sleeve is moved back.

The clamping sleeve can be displaced purely linearly. In this case, however, it must be ensured how the clamping sleeve is to be locked in the desired axial position. In a particularly advantageous refinement, the clamping sleeve involves a threaded sleeve which has a threaded portion which engages with a threaded portion of the endoscope head. The thread is preferably self-locking. The clamping sleeve can be a union nut. Preferably, however, the distal end of the endoscope head is formed by the damping bodies, while the clamping sleeve is displaced from the proximal to the distal end for clamping and thereby compresses the clamping bodies. The inner diameter of the clamping sleeve or threaded sleeve is preferably greater than the outer diameter of the radially inwardly displaced clamping bodies. If necessary, the clamping sleeve or threaded sleeve can be split in longitudinal direction, with the parts being connected to one another after assembly on the base body.

The force transmission from the clamping sleeve to the clamping bodies is realized according to the inclined plane principle. In this case, a normal force is exerted on a plane via a forwardly directed force. The force direction or the magnitude of the normal force depends on the angle of the inclined plane. For this purpose, the clamping sleeve itself can have an inclined, in particular conical, run-on surface. However, this is not necessarily required. The clamping bodies preferably have outer sides which already extend at an angle to the longitudinal axis of the longitudinal channel. Within the scope of the invention, it is possible to provide the clamping sleeve and/or the clamping bodies with suitable slanted surfaces in order to adjust a force transmission that matches the respective clamping situation.

There are applications, such as in bronchoscopy for example, which require provision of additional measuring channels in the endoscope tube (bronchoscope tube). One or more measuring channels have to accordingly feed into the endoscope head, in order to ascertain for example the pressure at the distal end of the endoscope tube via a measuring means which is connected to the endoscope head. In an advantageous refinement of the invention, provision is therefore made for the respective damping sleeve to have at least one channel with a radially outwardly open end and a radially inwardly open end. The inner end of the channel in the clamping sleeve is provided to be connected in a fluid-conducting manner to a channel in the damped state of the clamping device, with the channel being arranged in at least one damping body and being open radially inwards towards an endoscope tube to be received.

Because these two channels are positioned in the clamping situation in such a way that they are connected to one another in a fluid-conducting manner, a substantially radial passage from the outside to the inside can be created in the endoscope head, so that there is a connection between the measuring channel in the endoscope tube and a measuring instrument on the endoscope head.

As a consequence of the movable clamping sleeve and the movable clamping body, it is not trivial to place the individual channel openings in such a way that a fluid line is possible with the necessary certainty. Tight tolerances must be maintined. At the same time, it must be ensured that the clamping sleeve with its radially inner open end is arranged exactly above the outer end of the channel in the clamping body and of course the endoscope tube must asobe arranged in the correct angular position in the endoscope head. Preferably, corresponding markings or also mechanical detents are arranged on the endoscope head in order to ensure that only certain positions of the clamping sleeve are set in dependence on the diameter of the endoscope tube, namely exclusively those positions in which a fluid-conducting connection between the channels is possible.

In the case of clamping sleeves which are moved rotationally on the base body because they are mounted via a threaded portion, provision is made for arranging the channels in the clamping bodies offset with respect to one another in axial direction so as to conform to the pitch of the threaded portion. An example: An endoscope head is intended to receive two endoscope tubes of different thicknesses. It is known which positions the damping sleeves must assume in order to clamp the respective endoscope tube. In the first position for the thinner endoscope tube, the clamping sleeve must be displaced further in longitudinal direction, i.e., rotated further than for the thicker endoscope tube. Therefore, provision is made in a first clamping body for a channel, which is arranged closer to the distal end, and for a second channel in another clamping body, with the second channel being arranged not only in another circumferential region but also at a different distance from the distal end. The position of the channels in the clamping bodies is determined by the thread pitch and by the diameter of the used endoscope tubes.

According to a further advantageous configuration of the invention, the clamping sleeve includes at least one connection piece radially on the outside. This connection piece is intended to connect a measuring means. Therefore, the at least one channel, which passes radially through the clamping sleeve, should feed into the connection piece. It preferably feeds into the outer end thereof. The connection piece may serve at the same time as a display for the operator of the endoscope in order to check the correct position of the clamping body in relation to the diameter of the endoscope tube. At the same time, because of its length, the connection piece can be configured in such a way as to be configured as a lever and thereby to simplify the operability of the clamping sleeve. It must be dimensioned sufficiently large for this purpose so that it is not damaged even when the clamping sleeve is tensioned.

According to the invention, it is provided that the clamping device can be coupled to the endoscope tube in a fixed rotative engagement. The fixed rotative engagement can be established in a force-fitting and/or form-fitting manner by engaging at least a male piece on the endoscope tube and/or endoscope head with at least one female piece on the endoscope tube and/or on the endoscope head. For various applications, it is necessary to be able to rotate the endoscope tube into the desired position during use and also to be able to transmit a force to the distal end of the endoscope tube via the endoscope tube. This is possible in a most reliable manner via form-fitting connections. The form-fitting connection can be secured by the force-fitting connection. A form fit can be established through axial and/or radial engagement of the components which are in engagement with one another.

For this purpose, corresponding recesses as a female piece can be provided at the proximal end of the endoscope tube or in the end region of the endoscope tube, which recesses correspond to respective projections on the endoscope head (male piece).

According to a further embodiment of the invention, provision is made for several endoscope tubes, which can be coupled successively to the endoscope tube. In practical use, the diameter of the endoscope tubes that are used successively should preferably be increased. This case is described hereinafter, while it is noted that a use of the endoscope head from thick endoscope tubes to thinner endoscope tubes is possible. In any case, a first endoscope tube serves as a guide endoscope tube. In addition to the guide endoscope tube, at least one further endoscope tube is provided. The endoscope tubes can be made of dimensionally stable plastic. Each of the endoscope tubes has a working channel and is designed such as to be able to receive the first endoscope tube or a further endoscope tube with matching diameter coaxially or be received in such an endoscope tube. Therefore a set of endoscope tubes is involved, which are matched to one another in diameter. The endoscope tubes are provided to be inserted in succession preferably with increasing diameters of the working channels coaxially into a cavity subject to endoscopy. One of the further endoscope tubes can hereby be attached over an endoscope tube that is smaller in diameter, so that the endoscope tube that is respectively smaller in diameter is the guide endoscope tube for the further endoscope tube. The inner one of the endoscope tubes serving as a guide endoscope tube is designed, after attachment of the further endoscope tube, to be pulled out from the latter in order to clear the working channel of the further endoscope tube, with the respective outer endoscope tube being able to be coupled to the endoscope head. This presupposes a complete continuity of the working channel of the greater endoscope tube for the smaller endoscope tube arranged therein.

Such a device can include two to six endoscopes which are matched to one another. As a result of the greater cross-section with same material and same wall thickness, each subsequent, further endoscope has a greater flexural rigidity than the first endoscope. The endoscopes are preferably made of a semi-rigid or dimensionally stable plastic. It is possible to use endoscope tubes made of different materials. The endoscopes made of plastic can be made available as single-use endoscopes in order to be disposed of after the examination or treatment.

The use of the device according to the invention has the advantage that initially instruments can be used which have a small cross-section and are more flexible and that the cross-section can be incrementally increased without the need for a separate endoscope head for each further instrument. A quick-action coupling by way of the clamping elements acting in a clamping manner enables a rapid exchange and minimizes the treatment time. The device can in particular also be used in such a way that, if need be, a thicker endoscope tube can be changed to a thinner endoscope tube, e.g. when it turns out that a constriction cannot be passed with the thicker endoscope tube. In this case, the thicker endoscope tube serves as a guide endoscope for the thinner endoscope tube. In this procedure, the endoscope head can also be detached quickly and reliably from the respectively previously used endoscope tube and connected to the new endoscope tube. The method according to the invention is characterized by the following steps:
a) A first endoscope tube is inserted into a cavity subject to endoscopy and connected to the endoscope head;
b) A second endoscope tube of a diameter that deviates from the diameter of the first endoscope tube, is introduced coaxially to the first endoscope tube into the cavity, after the endoscope head has been removed from the first endoscope tube;
c) The first endoscope tube is removed and the endoscope tube remaining in the cavity is connected to the endoscope head. In this method, a greater endoscope tube is preferably placed over an endoscope tube that is smaller in diameter.

The invention will be explained hereinafter with reference to exemplary embodiments illustrated in purely schematic drawings. It is shown in:

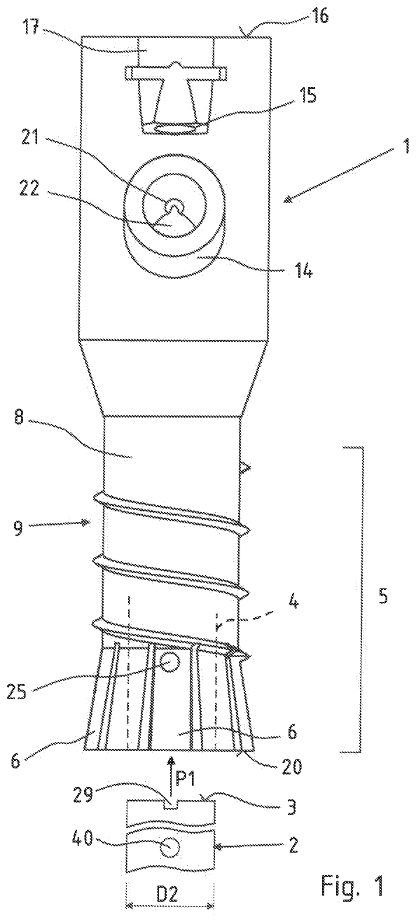

FIG. 1 a side view of an endoscope head;

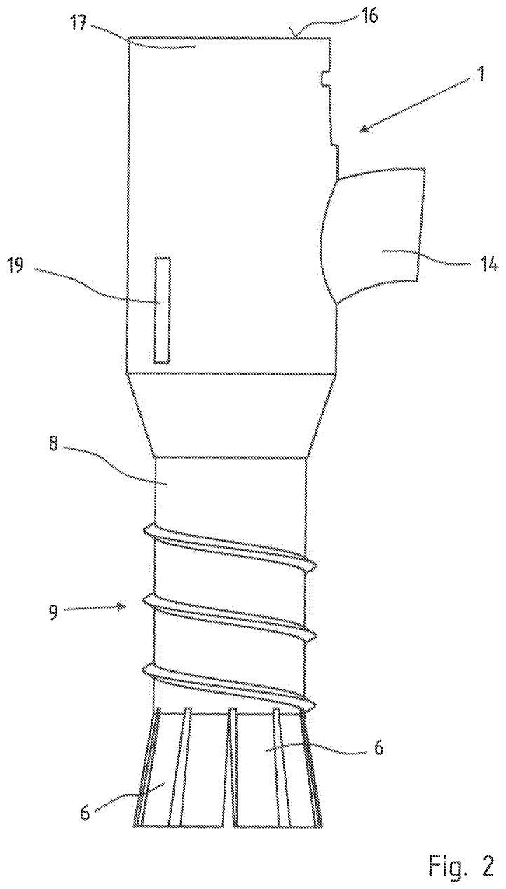

FIG. 2 a further side view of the endoscope head of FIG. 1;

FIG. 3 a perspective view of the endoscope head of FIG. 1;

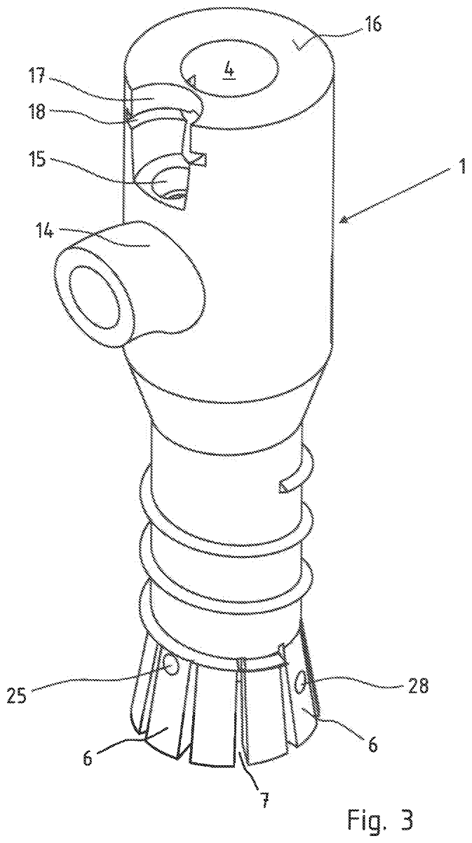

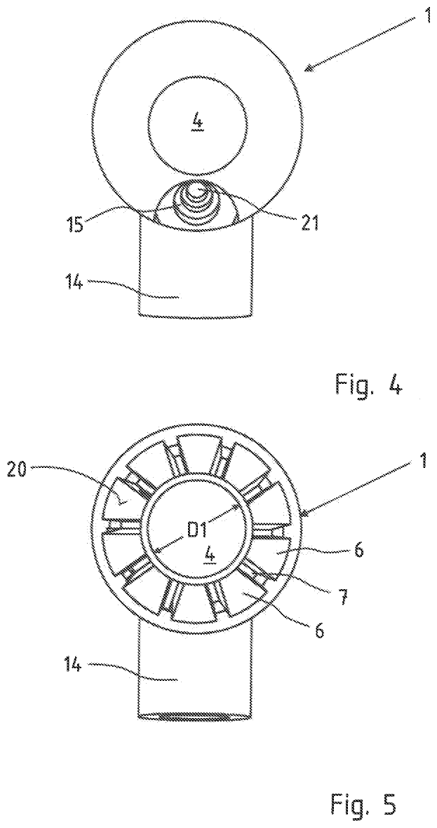

FIG. 4 a first end view onto the proximal end of the endoscope head of FIGS. 1 to 3;

FIG. 5 an end view upon the distal end of the endoscope head of FIGS. 1 to 4;

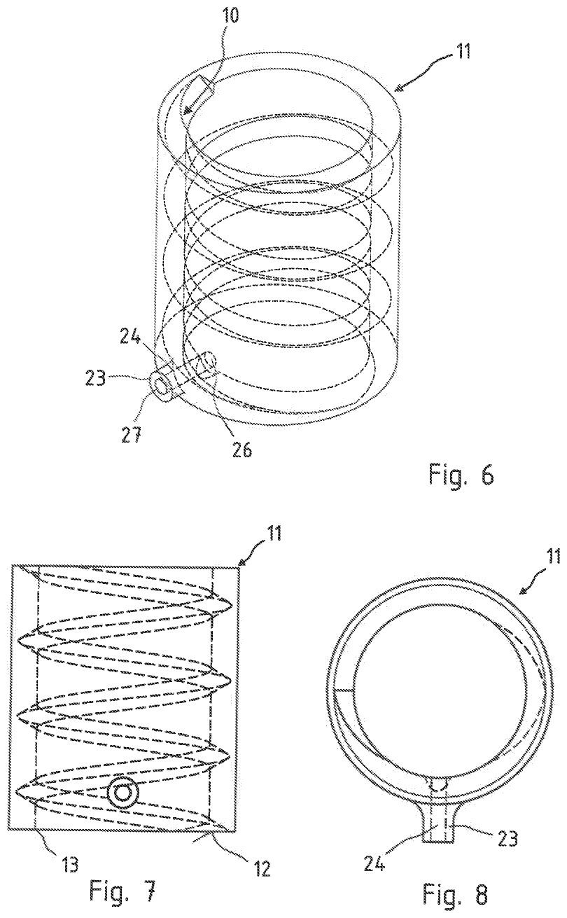

FIG. 6 a perspective view of a clamping sleeve;

FIG. 7 a side view of the clamping sleeve of FIG. 6;

FIG. 8 an end view of the clamping sleeve of FIGS. 6 and 7;

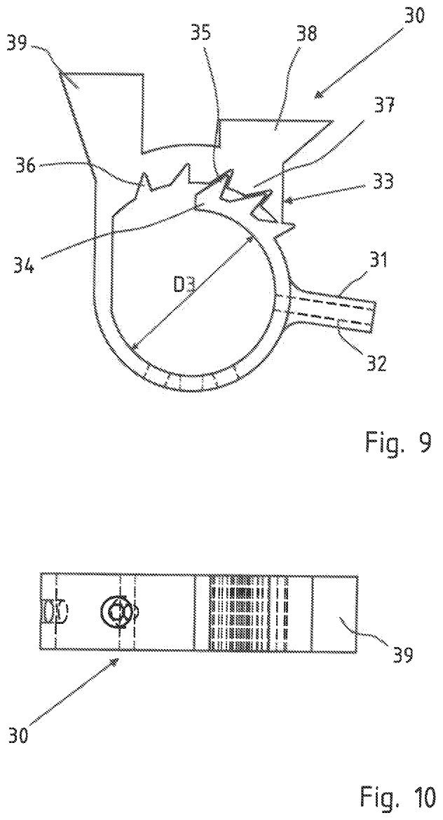

FIG. 9 a further embodiment of a clamping sleeve in a view upon its distal end;

FIG. 10 a view of the clamping sleeve of FIG. 9 from the side; and

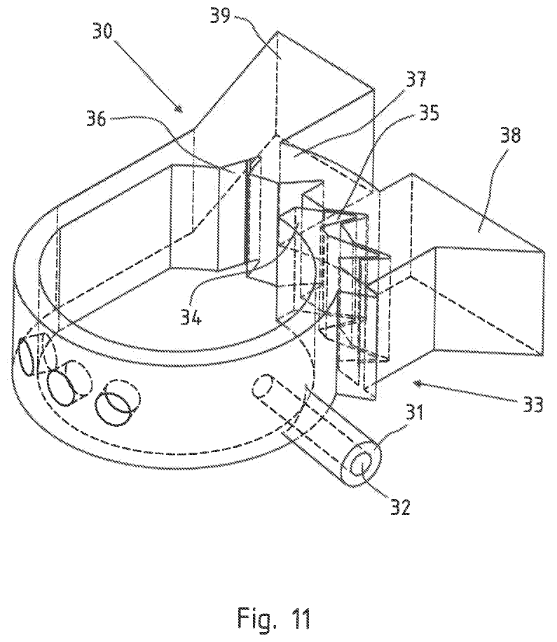

FIG. 11 a perspective view of the clamping sleeve of FIGS. 9 and 10.

FIG. 1 shows an endoscope head 1 as a component of a medical device. The same endoscope head 1 is also shown in various views in FIGS. 2 to 5.

The endoscope head 1 is designed to be connected to an endoscope tube 2. The endoscope tube 2 is shown only purely schematically and shortened in FIG. 1. FIG. 1 shows the proximal end of the endoscope tube 2. it is inserted in the direction of the arrow P1 into the distal end 20 of the endoscope head 1. For this purpose, a longitudinal channel 4 (FIGS. 4 and 5) is located within the endoscope head 1. The longitudinal channel 4 passes through the entire endoscope head 1. It serves as a working channel. The longitudinal channel 4 is indicated by a broken line in FIG. 1. In a manner not shown in greater detail, the longitudinal channel 4 can have graduations, e.g. in order to limit an attachment depth of an endoscope tube 2 to be received.

The endoscope head 1 includes a clamping device 5 having a functional portion that is marked in FIG. 1. The clamping device 5 includes several components. In particular, the clamping device 5 includes an arrangement of several displaceable clamping bodies 6. FIG. 5 shows an end view of the clamping bodies 6. They are arranged evenly dispersed over the circumference of the longitudinal channel 4. This exemplary embodiment involves 10 clamping bodies. In practice, there are at least three clamping bodies dispersed over the circumference, in particular evenly dispersed, in order to enable centering of an endoscope tube 2. The individual clamping bodies 6 are substantially wedge-shaped in cross section or circular segment-shaped due to the round, radially outer side and radially inner side. The individual clamping bodies 6 are configured identically. They are arranged at a distance to adjacent clamping bodies 6. Hence, a gap 7 is located between respective two clamping bodies 6. It has a constant width. This gap 7 can also be seen in the illustrations of FIGS. 1 to 3. It enables movement of the tongue-like clamping bodies 6 independently of one another and their displacement from outside to inside for clamping.

The individual clamping bodies 6 are connected to a base body 8. The base body 8 is cylindrical. In this exemplary embodiment, the base body 8 is a material-uniform component of the endoscope head 1. A threaded portion 9 is formed on the base body 8. The threaded portion 9 is provided for engagement with a threaded portion 10 on a clamping sleeve 11, as shown in FIGS. 6 to 9. The clamping sleeve 11 accordingly has an internal thread, while the threaded portion 9 on the base body 8 is an external thread.

Rotating the clamping sleeve 10 relative to the endoscope head or threaded portion 9 on the base body 8 causes the clamping sleeve 11 to be displaced in longitudinal direction of the endoscope head 1 and as a result to strike the clamping bodies 6. The clamping bodies 6 are configured in such a way that they clear an opening width D1, as shown in FIG. 5. The clamping bodies 6 assume an initial position in the Figures. They are relaxed. When the clamping sleeve 11 is now displaced in longitudinal direction toward the distal end 20 of the endoscope head 1 through rotation on the threaded portion 9, the distal end 12 of the clamping sleeve 11 strikes the individual clamping bodies 6. The clamping bodies 6 have a wedge-shaped cross section in longitudinal direction. In the initial position, they enclose a cylindrical interior, corresponding to the circular cross section of the endoscope tube 2 to be received. Radially on the outside, the several clamping bodies 6 are of frustoconical configuration in the sense that each individual outer surface of the clamping bodies 6 describes a frustoconical outer segment. The outer diameter of the circle spanned by the clamping bodies 6 is greatest at the distal end of the endoscope head 1 and corresponds at the proximal end of the clamping bodies 6 to the outer diameter of the cylindrical base body 8. A radially inner edge of the distal end 12 of the clamping sleeve 11 serves as a run-on surface 13 (FIG. 7). This annular run-on surface 13 is the surface on which the individual clamping sleeves 6 are supported and via which the damping sleeves 6 can be actively displaced inwards or displaced outwards by springing back when the damping sleeve is rotated back. As a result, it is possible to adjust the variable opening width D1 in such a way that endoscope tubes 2 of different diameters D2 can be received in the longitudinal channel 4 and above all held by the clamping device 5.

Following the base body 8 or following the clamping device 5, the endoscope head 1 includes a radially slantingly extending connection piece 14 for a ventilator towards its proximal end 16. The connection piece 14 is longitudinally channelized and feeds into the longitudinal channel 4. The connection piece 14 is illustrated symbolically.

Furthermore, the endoscope head 1 includes a second connection 15 for jet ventilation. This connection 15, when viewed in longitudinal direction, lies flush behind the connection piece 14 for ventilation. The connection 15 feeds into a channel which runs at an acute angle to the longitudinal axis of the endoscope head 1 and thus to the longitudinal channel 4. The connection 15 therefore also feeds at the end side parallel to the longitudinal channel 4 in the proximal end 16. The connection 15 itself is situated in a radial depression 17, which is introduced from the outside. This radially and axially open depression 17 at the proximal end 16 is arranged upstream of the actual connection 15 or the channel for jet ventilation. A groove 18 extending transversely to the longitudinal axis is located in this depression. A locking body (not shown) can be inserted into this groove 18. The locking body represents a barrier which would have to be actively removed in order to carry out jet ventilation. This is intended to prevent ventilation via the connection piece 14 during bronchoscopy and at the same time jet ventilation via the connection 15. The locking body, not shown in more detail, can be accommodated in a pocket 19, as can be seen in FIG. 2, when not in use. The pocket 19 is located on the side facing away from the connection piece 14 or the connection 15 for the jet ventilation, i.e, diametrically to the connection piece 14.

FIGS. 4 and 5 show the substantially cylindrical structure of the endoscope head 1, once with the viewing direction onto its proximal end 16 and once with the viewing direction on its distal end 20. FIG. 4 also shows a channel 21 for jet ventilation within the connection 15. The channel 21 for jet ventilation feeds into the ventilation channel 22 in the connection piece 14, as shown in FIG. 1. The channel 21 feeds quasi into the transition zone to the longitudinal channel 4 of the endoscope head 1.

The clamping body 11, as shown in FIGS. 6 to 8, includes a connection piece 23. The connection piece 23 is located adjacent to the distal end 12 and protrudes radially. Extending within the connection piece 23 is a channel 24. The channel 24 extends radially from the inside to the outside. Depending on the position of the clamping body 11 on the threaded portion 9 of the base body 8, the connection piece 23 and thus also the channel 24 arranged therein are located in different positions. The connection piece 23 serves for fluid passage, in particular for gas measurement in conjunction with an endoscope tube 2, which is accommodated in the endoscope head 1. For this purpose, a measuring opening 40 is located in the endoscope tube 2. This measuring opening 40 is so positioned during insertion of the endoscope tube 2 that the measuring opening 25 is connected in a fluid-conducting manner to a channel 25 which is located in one of the clamping bodies 6. The channel 25 in the damping body 6 must in turn be connected in a fluid-conducting manner to the channel 24 in the connection piece 23 of the clamping sleeve 11. For this purpose, the clamping sleeve 11 is rotated such that the radially inner open end 26 of the channel 24 is brought into overlap with the channel 25 in the clamping body 6. A measuring device can then be connected to the radially outer open end 28 of the channel 24 in the connection piece 23, for example to measure the air pressure.

Due to the fact that, in the case of different diameters of the endoscope tubes 2, the clamping sleeve 11 is also arranged in different positions, provision must be made for corresponding channels 25, 28 in the clamping bodies 6. FIG. 3 shows arrangement of several channels 25, 28 in different axial length portions of the clamping bodies 6. This ensures that channel 24 in the clamping sleeve 11 can be brought into fluid-conducting connection with a channel 25, 28 in the clamping bodies 6.

In order to ensure that the endoscope tube 2 is arranged in the correct position with respect to the measuring opening, a form-fitting means is arranged at the proximal end of the endoscope tube 2 for positioning. It can be designed as a male piece or a female piece. In this case, it is a female piece 29 in the form of a depression in the proximal end of the endoscope tube, as shown by way of example in FIG. 1. This female piece 29, which corresponds inside the endoscope head 1 to a male piece, not shown in more detail, can further ensure a fixed rotative engagement in order to hold the endoscope tube 2 not only in a force-fitting manner through clamping in the endoscope head 1, but also in a form-fitting manner. The attachment depth is defined in the endoscope head 1 and thus the exact position of the measuring opening 40 relative to the channel 25 in the clamping body 6. In addition, a torque can be exerted on the endoscope tube 2 via the endoscope head 1.

FIGS. 9 to 11 show an alternative clamping sleeve 30. This design of the damping sleeve 30 can also be used with an endoscope head 1, as shown in FIGS. 1 to 5. The clamping sleeve 30 again has a connection piece 31 with a channel 32 analogously to the connection piece 23 with the channel 24 of the damping sleeve 11 according to FIG. 8. The essential difference according to the clamping sleeve explained first (FIGS. 6 to 8) is that this clamping sleeve 30 does not have a constant inner diameter, but that the diameter D3 is variable. Due to the fact that the clamping sleeve 30 is not displaced in longitudinal direction, the channel 32 remains in the connection piece 23 at the same spot. Care must merely be taken to ensure that the clamping sleeve 30 of this type is placed in the correct length portion. For this purpose, corresponding positioning aids may be provided on the clamping bodies 6.

The clamping bodies 6 are compressed when the diameter D3 of the clamping ring is reduced and are correspondingly liberated when the diameter D3 is increased. The damping sleeve 30 has a locking device 33 which makes it possible to adjust the clamping sleeve 30 to predefined, different diameters. For this purpose, radially outwardly directed latching lugs 35 are located on an inner end portion 34 of the clamping sleeve 33. This example involves three identical latching lugs 35. These latching lugs 35 are provided to engage in latching recesses 36 of an outer end portion 37 of the clamping sleeve 30. Two latching lugs 35 are in engagement with two latching recesses 36. This results in three differently adjustable diameter regions in the geometry selected here. When it is sufficient for only one latching lug 35 to be in engagement with a latching recess 36, there are even four adjustable diameters which can be selected by adjusting the latching positions.

The adjustment of the diameters towards smaller diameter regions is simplified by beveling the latching lugs 35 and also the latching exceptions 36 on one side, corresponding to a sawtooth design. The steeper flanks of the latching lugs 35, which in particular extend radially to the longitudinal axis of the clamping sleeve 30, prevent inadvertent opening.

A first handling portion 38 is located at the radially outer end portion 37 and simplifies gripping of the outer end portion 37 of the clamping sleeve 30 in order to release the clamping sleeve 30. Another radially outwardly projecting handling portion 39 is located at a distance from the first handling portion 38 and serves for handling during the clamping of the clamping sleeve 30.

Further details of the clamping sleeve 30 can be seen with reference to FIGS. 10 and 11.

### REFERENCE SIGNS

1—endoscope head

2—endoscope tube

3—proximal end of 2

4—longitudinal channel of 1

5—clamping device

6—damping body

7—channel between 6

8—base body

9—threaded portion at 8

10—threaded portion at 11

11—clamping sleeve

12—distal end of 11

13—run-on surface at 11

14—connection piece

15—connection for jet ventilation

16—proximal end of 1

17—depression in 1

18—groove in 17

19—pocket in 1

20—distal end of 1

21—channel for jet ventilation

22—channel for ventilation

23—connection piece

24—channel in 23

25—channel in 6

26—inner end of 24

27—outer end of 24

28—channel in 6

29—female piece in 2

30—clamping sleeve

31—connection piece

32—channel in 31

33—locking means

34—inner end portion of 30

35—latching lug at 34

36—latching recess at 37

37—outer end portion of 30

38—first handling means at 30

39—second handling means

40—measuring opening in 2

D1—opening width of 4

D2—diameter of 2

D3—diameter of 30

P1—arrow

1.-14. (canceled)

15. A medical device, comprising an endoscope head designed to be detachably connected to endoscope tubes of different diameters and including a longitudinal channel for receiving a respective one of the endoscope tubes, said longitudinal channel having an opening width which is variable to receive the endoscope tubes of different diameters, said endoscope head including a clamping device designed to hold the respective one of the endoscope tubes inserted into the longitudinal channel in a clamping manner, said clamping device including a base body, a clamping sleeve having a threaded portion in engagement with a threaded portion of the base body so as to be displaceable axially with respect to the base body, and a plurality of displaceable clamping bodies which are movably connected to the base body and dispersed over a circumference of the longitudinal channel such as to be able to be urged from radially outwards against the respective one of the endoscope tubes, when the clamping sleeve is rotated relative to the endoscope head and displaced in a longitudinal direction.

16. The medical device of claim 15, wherein the clamping bodies are in operative engagement with a run-on surface of the clamping sleeve.

17. The medical device of claim 15, wherein the clamping sleeve has an inner diameter which is variable in a contact region with the clamping bodies.

18. The medical device of claim 15, wherein the clamping sleeve includes a channel having a radially outwardly open end and a radially inwardly open end, said radially inwardly open end being in fluid communication with a channel which is arranged in at least one of the clamping bodies and open radially inwards in relation to the respective one of the endoscope tubes, when the clamping device is in a clamped state.

19. The medical device of claim 18, wherein the radially inwardly open end of the channel of the clamping sleeve is in fluid communication with channels arranged in the damping bodies, with the channels being arranged offset with respect to one another in an axial direction of the longitudinal channel in conformity with a pitch of the threaded portion of the base body.

20. The medical device of claim 18, wherein the damping sleeve includes a connection piece radially on an outside, with the channel of the clamping sleeve feeding into the connection piece.

21. The medical device of claim 15, wherein the damping bodies are firmly connected to the endoscope head.

22. The medical device of claim 15, wherein the endoscope head is coupleable to the respective one of the endoscope tubes in fixed rotative engagement, with the fixed rotative engagement designed to be force-fittingly and/or form-fittingly by bringing a male piece on the respective one of endoscope tubes and/or on the endoscope head into engagement with a female piece on the respective one of the endoscope tubes and/or on the endoscope head.

23. The medical device of claim 15, wherein a first one of the endoscope tubes represents a guide endoscope tube and at least a further one of the endoscope tubes has a working channel and is designed to receive coaxially the first one of the endoscope tubes or a still further one of the endoscopes tube fitting in diameter or to be received in the still further one of the endoscope tubes, said endoscope tubes being designed for successive introduction into a cavity subject to endoscopy and attachable to one another in such a way that the first one of the endoscope tubes is a guide for the further one of the endoscope tubes of greater diameter when being attached, said guide endoscope tube being retracted after coaxial introduction of the further one of the endoscope tubes which is coupled to the endoscope head.

24. An endoscopic method, comprising:
inserting a first endoscope tube into a cavity subject to endoscopy;
introducing a second endoscope tube of a diameter which deviates from a diameter of the first endoscope tube in coaxial relationship to the first endoscope tube into the cavity;
removing the first endoscope tube; and
connecting the second endoscope tube to an endoscope head of a medical device as set forth in claim 15.

25. The endoscopic method of claim 24, wherein the diameter of one of the first and second endoscope tubes is greater that the diameter of the other one of the first and second endoscope tubes, and further comprising pushing the endoscope tube of greater diameter over the endoscope tube of smaller diameter.
